(19) United States
(12) Patent Application Publication
(10) Pub. No.: US 2014/0364728 A1
(43) Pub. Date: Dec. 11, 2014

(54) Insertion Target Point Setting Apparatus, Ultrasound Diagnostic Apparatus and Method for Setting Insertion Target Point

(71) Applicant: GE MEDICAL SYSTEMS GLOBAL TECHNOLOGY COMPANY, LLC, Waukesha, WI (US)

(72) Inventor: Hiroshi Hashimoto, Tokyo (JP)

(73) Assignee: GE MEDICAL SYSTEMS GLOBAL TECHNOLOGY COMPANY, LLC, Waukesha, WI (US)

(21) Appl. No.: 14/299,337

(22) Filed: Jun. 9, 2014

(30) Foreign Application Priority Data
Jun. 11, 2013 (JP) 2013123068

(51) Int. Cl. A61B 8/08; A61B 10/02

(52) U.S. Cl. CPC A61B 8/0841; A61B 10/0233
USPC 600/437

(57) ABSTRACT

An insertion target point setting apparatus is provided. The insertion target point setting apparatus includes a reference point setting unit configured to set, in coordinates formed in a three-dimensional space with a subject with a biopsy needle inserted therein, a first reference point which is an insertion target point of the biopsy needle in the subject, and a second reference point which is a noticed portion indication point indicative of a noticed portion in the subject, and an insertion target point setting unit configured to set other insertion target points other than the first reference point in the coordinates formed in the three-dimensional space based on the first reference point and the second reference point, using a predetermined drawing method.

### CROSS REFERENCE TO RELATED APPLICATIONS

This application claims the benefit of Japanese Patent Application No. 2013-123068 filed Jun. 11, 2013, which is hereby incorporated by reference in its entirety.

### BACKGROUND OF THE INVENTION

The present invention relates to an insertion target point setting apparatus for setting insertion target points taken as targets in each of which a biopsy needle is inserted, an ultrasound diagnostic apparatus, and a method for setting insertion target points.

In an ultrasound diagnostic apparatus, an ultrasound image of a subject can be displayed in real time. Thus, when a biopsy needle is inserted in the subject, confirming by an ultrasound image whether or not the biopsy needle has been inserted up to the position of its ablation has been carried out (refer to, for example, Japanese Unexamined Patent Publication No. 2012-245092).

Meanwhile, there is a case where a plurality of biopsy needles are inserted in the periphery of a lesion such as a tumor to carry out its ablation. In this case, the lesion may not be ablated thoroughly with a deviation in the position of the biopsy needle. It is thus desirable that a plurality of biopsy needles are inserted to sufficiently ablate the lesion thoroughly and avoid a deviation in the position relative to the lesion. For that purpose, it is desirable that insertion targets can be set where the plural biopsy needles are inserted.

### BRIEF DESCRIPTION OF THE INVENTION

In a first aspect, an insertion target point setting apparatus is provided. The insertion target point setting apparatus includes a reference point setting unit which sets in coordinates formed in a three-dimensional space with a subject with a biopsy needle inserted therein being present therein, a first reference point which is an insertion target point of the biopsy needle in the subject, and a second reference point which is a noticed portion indication point indicative of a noticed portion in the subject, and an insertion target point setting unit which sets other insertion target points other than the first reference point in the coordinates formed in the three-dimensional space on the basis of the first reference point and the second reference point using a predetermined drawing method.

In a second aspect, an insertion target point setting apparatus according to the first aspect is provided, in which the insertion target point setting unit sets based on the first reference point and the second reference point, a regular polygon of which apexes are located around the noticed portion and having as one apex, the insertion target point being the first reference point, and sets above other insertion target points to other apexes of the regular polygon.

In a third aspect, an ultrasound diagnostic apparatus including the insertion target point setting apparatus according to the first aspect is provided. The ultrasound diagnostic apparatus is equipped with an indicator display control unit that causes an indicator to be displayed in a position of each of the insertion target points in the ultrasound image of the subject.

In a fourth aspect, an ultrasound diagnostic apparatus including the insertion target point setting apparatus according to the first aspect is provided. The ultrasound diagnostic apparatus is equipped with a distance indicator display control unit that causes a distance indicator indicative of a distance between each of the insertion target points and a transmission/reception plane of ultrasound to and from the subject to be displayed on the ultrasound image of the subject.

According to the first aspect, when the first reference point and the second reference point are set, other insertion target points are set by a predetermined drawing method on the basis of these. It is therefore possible to set insertion targets where a plurality of biopsy needles are inserted.

Further, according to second aspect referred to above, the apexes of a regular polygon are set around the noticed portion, and the insertion target points are set to the apexes thereof. Therefore, if biopsy needles are inserted into the insertion target points, the biopsy needles can be inserted in such a manner that their positions are not deviated.

According to the third aspect referred to above, since an indicator is displayed in the position of each of the insertion target points in the ultrasound image, it is possible to insert each biopsy needle toward the indicator.

According to the fourth aspect, since a distance indicator indicating the distance between each of the insertion target points and the transmission/reception plane of ultrasound to and from the subject is displayed, it is possible to easily display an ultrasound image including each of the insertion target points.

Further advantages will be apparent from the following description of the exemplary embodiments as illustrated in the accompanying drawings.

### BRIEF DESCRIPTION OF THE DRAWINGS

FIG. 1 is a block diagram showing one example of a schematic configuration of an ultrasound diagnostic apparatus according to a first embodiment and an RFA device.

FIG. 2 is a block diagram illustrating a configuration of a display controller in the ultrasound diagnostic apparatus according to the first embodiment.

FIG. 3 is a diagram for describing the manner in which three biopsy needles are inserted.

FIG. 4 is an enlarged diagram showing the points of the biopsy needles.

FIG. 5 is a flow chart showing a process for setting biopsy target points.

FIG. 6 is a diagram illustrating a display unit on which an ultrasound image is displayed.

FIG. 7 is a diagram showing the display unit being in a state in which a marker is set to a lesion.

FIG. 8 is a diagram for describing the setting of the marker using the biopsy needle.

FIG. 9 is a diagram illustrating the display unit in which a marker is displayed in a position of an intersection between an extension line of the biopsy needle and a plane.

FIG. 10 is a diagram showing markers Mt and Mn, a first reference point pb1 and a second reference point pb2 in a plane P.

FIG. 11 is a diagram depicting an equilateral triangle set to the plane P shown in FIG. 10, and a first insertion target point, a second insertion target point and a third insertion target point set to the apexes of the equilateral triangle.

FIG. 12 is a diagram showing a circle of which the radius is defined between the first reference point and the second reference point and the center is defined as the second reference point.

FIG. 13 is a diagram for describing that the second insertion target point and the third insertion target point are set on the circumference of the circle shown in FIG. 12.

FIG. 14 is a diagram showing a plane which passes through the first insertion target point, a plane which passes through the second insertion target point, and a plane which passes through the third insertion target point.

FIG. 15 is a diagram illustrating the display unit on which a first distance indicator, a second distance indicator and a third distance indicator are displayed.

FIG. 16 is a diagram showing an ultrasound image in a state in which the first insertion target point exists on an ultrasound transmission/reception plane.

FIG. 17 is a diagram depicting an ultrasound image in a state in which a biopsy needle is inserted into a subject.

FIG. 18 is a block diagram showing one example of a schematic configuration of an ultrasound diagnostic apparatus according to a second embodiment and an RFA device.

FIG. 19 is a block diagram illustrating a configuration of a display controller in the ultrasound diagnostic apparatus according to the second embodiment.

### DETAILED DESCRIPTION OF THE INVENTION

Exemplary embodiments will hereinafter be described.

### First Embodiment

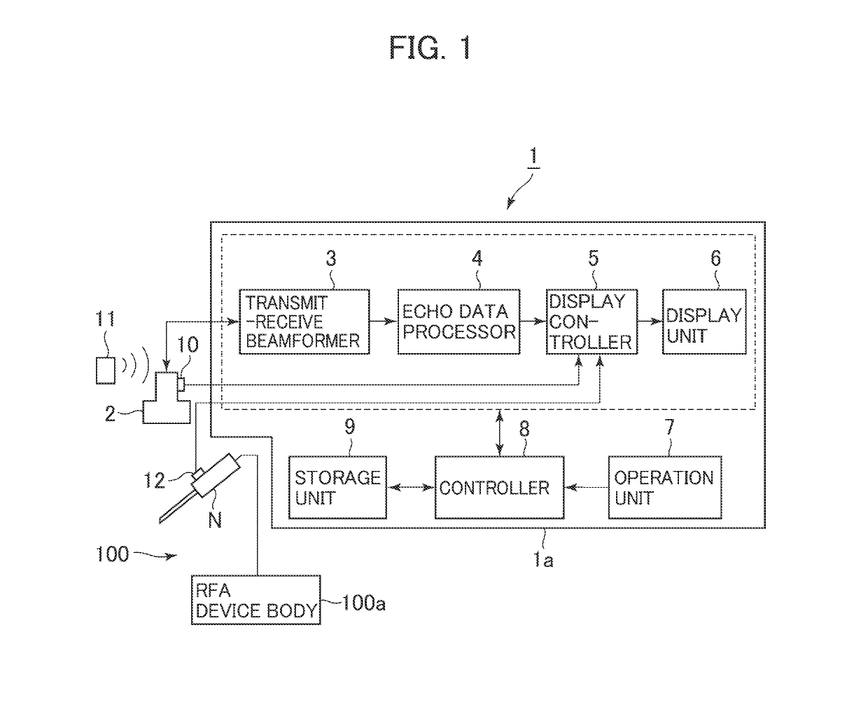

An ultrasound diagnostic apparatus 1 shown in FIG. 1 is equipped with an ultrasound probe 2, a transmit-receive beamformer 3, an echo data processor 4, a display controller 5, a display unit 6, an operation unit 7, a controller 8 and a storage unit 9. The transmit-receive beamformer 3, the echo data processor 4, the display controller 5, the display unit 6, the operation unit 7, the controller 8 and the storage unit 9 are provided in an apparatus body 1a of the ultrasound diagnostic apparatus 1. The apparatus body 1a and the ultrasound probe 2 are connected to each other through a cable. The ultrasound diagnostic apparatus 1 includes an insertion target point setting apparatus.

The ultrasound probe 2 includes a plurality of ultrasound transducers (not shown) arranged in an array form. The ultrasound probe 2 transmits ultrasound to a subject through the ultrasound transducers and receives its echo signals therein. The ultrasound probe 2 performs the transmission/reception of the ultrasound to and from a three-dimensional region of the subject to obtain volume data as will be described later.

The ultrasound probe 2 is provided with a first magnetic sensor 10 including, for example, a Hall element. The first magnetic sensor 10 detects magnetic field generated from a magnetic field generating unit 11 including, for example, a magnetic field generating coil. Coordinates are formed in a three-dimensional space by the magnetic field generating unit 11. Accordingly, the coordinates by the magnetic field generating unit 11 are formed even in a three-dimensional region of the subject being in the three-dimensional space.

A detection signal from the first magnetic sensor 10 is inputted to the display controller 5. The detection signal from the first magnetic sensor 10 may be inputted to the display controller 5 through a cable or may be inputted to the display controller 5 wirelessly. The magnetic field generating unit 11 and the first magnetic sensor 10 are provided to detect the position and tilt of the ultrasound probe 2 as will be described later.

The transmit-receive beamformer 3 supplies an electric signal for transmitting ultrasound from the ultrasound probe 2 under a predetermined scan condition to the ultrasound probe 2 on the basis of a control signal from the controller 8. Further, the transmit-receive beamformer 3 performs signal processing such as A/D conversion, phasing-adding processing and the like on each echo signal received by the ultrasound probe 2 and outputs echo data subsequent to the signal processing to the echo data processor 4.

The echo data processor 4 performs processing for generating an ultrasound image on the echo data outputted from the transmit-receive beamformer 3. For example, the echo data processor 4 performs B-mode processing such as logarithmic compression processing and envelope detection processing or the like to generate B-mode data.

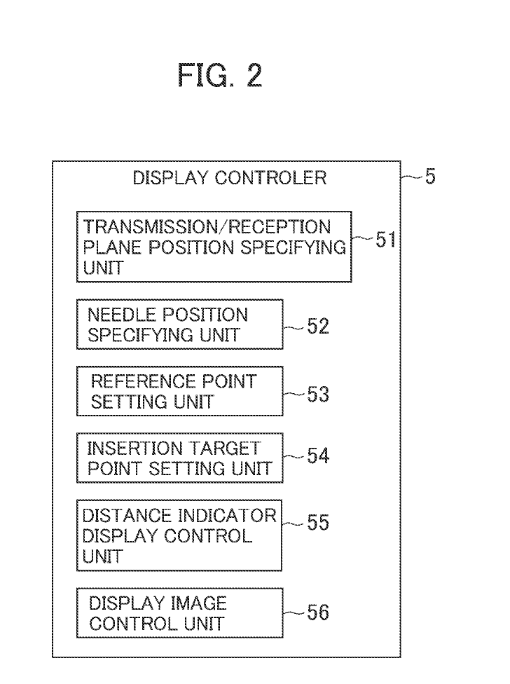

The display controller 5 has a transmission/reception plane position specifying unit 51, a needle position specifying unit 52, a reference point setting unit 53, an insertion target point setting unit 54, a distance indicator display control unit 55 and a display image control unit 56 as shown in FIG. 2. The transmission/reception plane position specifying unit 51 first calculates information (hereinafter called “probe position information”) about the position and tilt of the ultrasound probe 2 in a coordinate system of a three-dimensional space with the magnetic field generating unit 11 taken as an origin, based on the magnetic detection signal from the first magnetic sensor 10. Next, the transmission/reception plane position specifying unit 51 calculates information about the position of each echo signal in the coordinate system of the three-dimensional space, based on the probe position information. Thus, the position of a transmission/reception plane of the ultrasound in the coordinates of the three-dimensional space is specified.

The needle position specifying unit 52 specifies the position and direction of a biopsy needle N in the coordinate system of the three-dimensional space with the magnetic field generating unit 11 taken as the origin. Described more specifically, the biopsy needle N is provided with a second magnetic sensor 12 including, for example, a Hall element. The second magnetic sensor 12 detects magnetic field generated from the magnetic field generating unit 11. A detection signal from the second magnetic sensor 12 is inputted to the display controller 5. The needle position specifying unit 52 specifies the position and direction of the biopsy needle N in the coordinate system of the three-dimensional space with the magnetic field generating unit 11 taken as the origin, based on the magnetic detection signal from the second magnetic sensor 12.

Incidentally, the biopsy needle N is a needle used in RFA (RadioFrequency Ablation) and applies a radiofrequency. The biopsy needle N is connected to an RFA device body 100a. An RFA device 100 includes the biopsy needle N and the RFA device body 100a. The RFA device body 100a controls the irradiation of the radiofrequency from the biopsy needle N.

Incidentally, only one biopsy needle N is illustrated in FIG. 1, but in the first embodiment, ablation is performed in a state in which a plurality of the biopsy needles N are inserted. Thus, the biopsy needles are connected to the RFA device body 100a in plural form.

Let's return to the description of the display controller 5. The reference point setting unit 53 sets a first reference point pb1 being a first insertion target point pt1 of the biopsy needle N in the subject, and a second reference point pb2 being a noticed portion indication point pn indicative of a portion to be noticed in the subject. The first insertion target point pt1 is a point taken to be an insertion target of the biopsy needle N in the subject. Further, the portion to be noticed in the subject is a lesion, for example. The noticed portion indication point pn is set to the lesion of the subject.

The first reference point pb1 and the second reference point pb2 are set in the coordinates formed in the three-dimensional space. Their details will be described later.

The insertion target point setting unit 54 sets other insertion target points (second insertion target point pt2 and third insertion target point pt3 to be described later) other than the first insertion target point pt1 on the basis of the first reference point pb1 and the second reference point pb2. Their details will be described later.

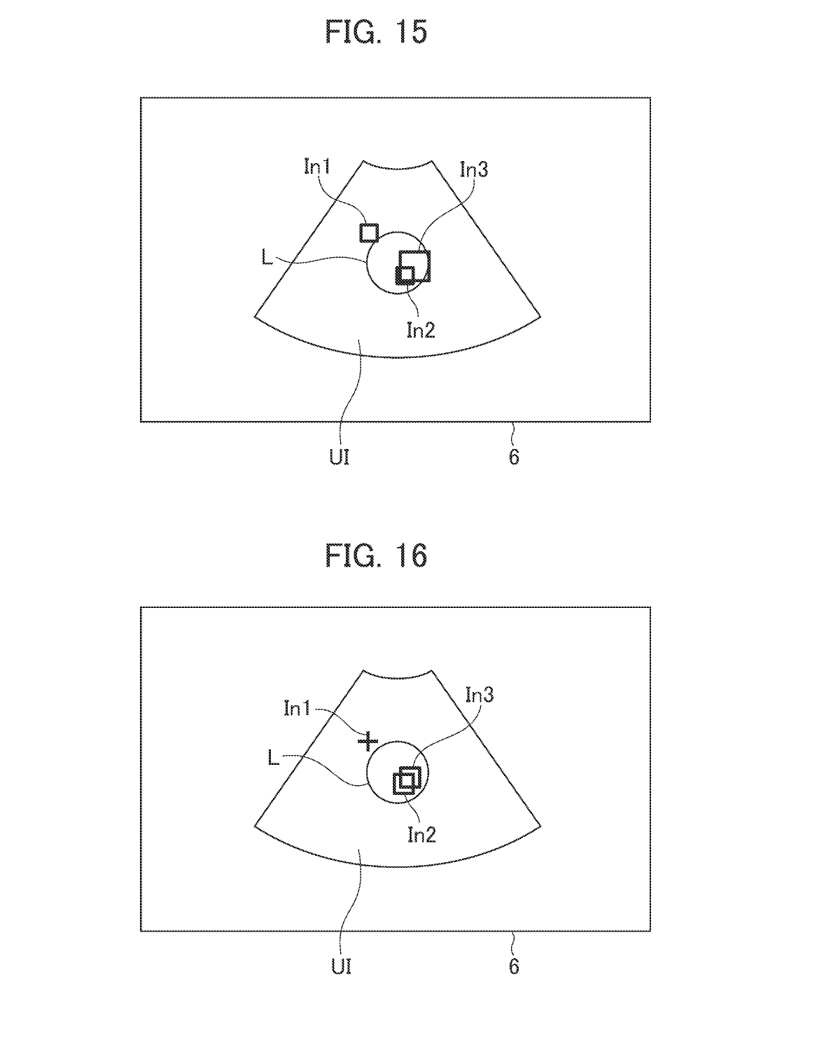

The distance indicator display control unit 55 causes the display unit 6 to display distance indicators In (refer to a first distance indicator In1, a second distance indicator In2 and a third distance indicator In3, FIG. 15 and the like to be described later) each indicative of the distance between each of the insertion target points pt1 through pt3 and the ultrasound transmission/reception plane. The details thereof will be described later.

The display image control unit 56 performs scan conversion on data inputted from the echo data processor 4 by a scan converter to generate ultrasound image data. Further, the display image control unit 56 causes the display unit 6 to display an ultrasound image based on the ultrasound image data. The ultrasound image is a B-mode image, for example.

The display unit 6 is an LCD (Liquid Crystal Display), an organic EL (Electro-Luminescence) display or the like.

The operation unit 7 includes, although not illustrated in particular, a keyboard for inputting instructions and information by an operator, a pointing device such as a trackball, etc.

The controller 8 has a CPU (Central Processing Unit) although not illustrated in particular. The controller 8 reads a control program stored in the storage unit 9 to execute functions at the respective parts of the ultrasound diagnostic apparatus 1.

The storage unit 9 is a semiconductor memory such as an HDD (Hard Disk Drive), a RAM (Random Access Memory), a ROM (Read Only Memory) or the like.

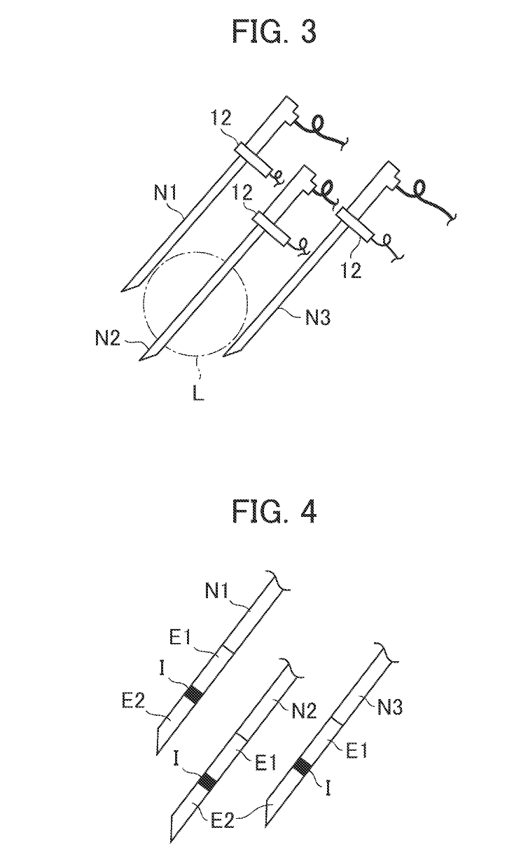

The operation of the ultrasound diagnostic apparatus 1 according to the first embodiment will now be described. In the first embodiment, as shown in FIG. 3, three biopsy needles N1, N2 and N3 are inserted in a subject P, and radiofrequency-based ablation is carried out. The three biopsy needles N1, N2 and N3 are inserted in the periphery of a lesion L such as a tumor.

Incidentally, the points of the biopsy needles N1 through N3 are respectively provided with a first electrode E1 and a second electrode E2 as shown in FIG. 4. An insulator I insulates between these first and second electrodes E1 and E2.

The biopsy needles N1, N2 and N3 are inserted in the subject in such a manner that the range of ablation by the biopsy needles N1 through N3 includes the lesion L. The biopsy needles N1 through N3 may be inserted so as to avoid a deviation in the ablation range and to be parallel to each other and pass through the apexes of the equilateral triangle (thus, the biopsy needles N1 through N3 are positioned in the corner parts of a triangular column (not shown)). In the first embodiment, before the biopsy needles N1 through N3 are inserted, their biopsy target points are set in such a manner that these biopsy needles N1 through N3 are inserted so as to pass through the apexes of the equilateral triangle.

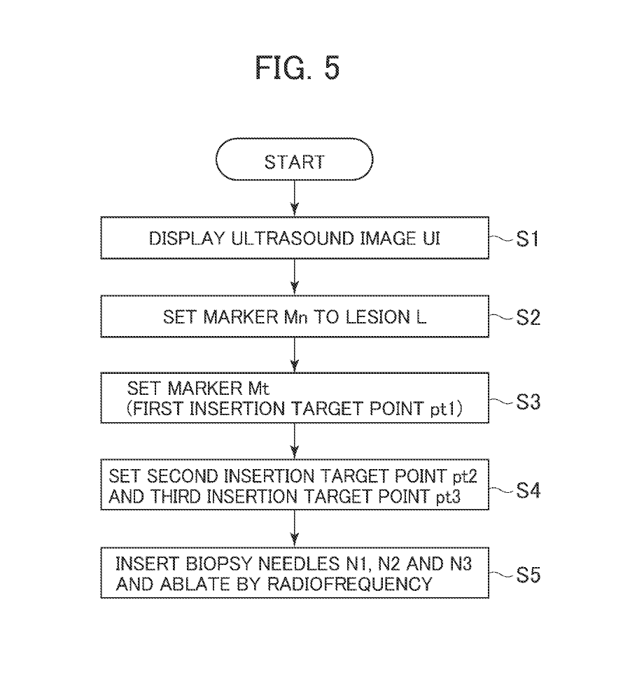

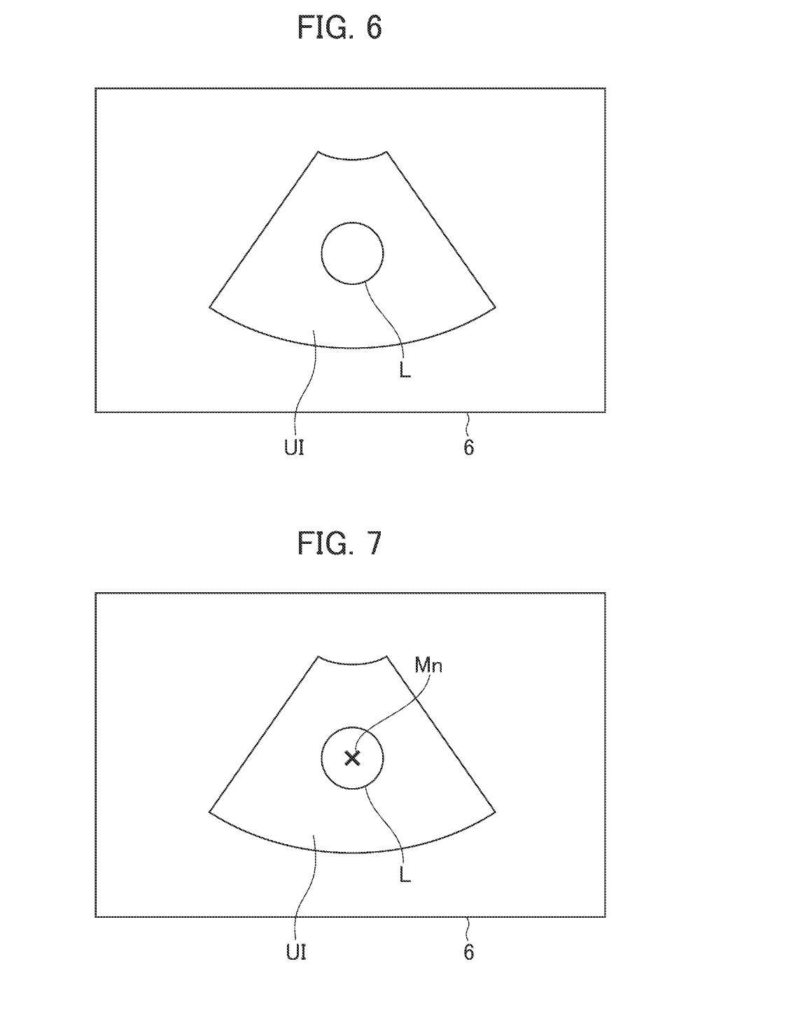

The above setting process will specifically be described based on the flow chart shown in FIG. 5. First, at Step S1, an ultrasound image UI is displayed on the display unit 6 as shown in FIG. 6. The operator performs the transmission/reception of ultrasound by the ultrasound probe 2 brought into contact with the body surface of the subject P. The ultrasound image UI based on echo signals of the ultrasound, which are obtained by the ultrasound probe 2, is displayed on the display unit 6. The operator positions the ultrasound probe 2 in such a manner that a lesion L is displayed in the ultrasound image UI and performs the transmission/reception of the ultrasound.

Next, at Step S2, the operator sets a marker Mn to the lesion L displayed in the ultrasound image UI using the track ball or the like of the operation unit 7 as shown in FIG. 7. The reference point setting unit 53 sets the marker Mn, based on a signal inputted from the operation unit 7. A point to which the marker Mn is set is a noticed portion indication point pn, which is a second reference point pb2.

The reference point setting unit 53 specifies the coordinates of the marker Mn in the coordinate system of the three-dimensional space. The reference point setting unit 53 specifies the coordinates of the marker Mn in the coordinate system of the three-dimensional space from the position of an ultrasound transmission/reception plane, which is specified by the transmission/reception plane position specifying unit 51, and the position of the marker Mn in the ultrasound image UI. The coordinates of the marker Mn may be stored in the storage unit 9.

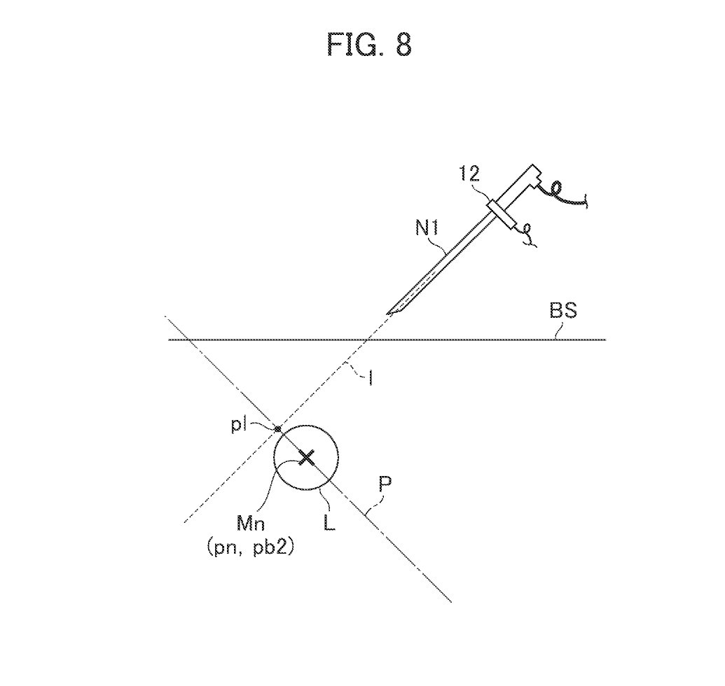

Next, at Step S3, the reference point setting unit 53 set a marker Mt to a point which serves as an insertion target for the biopsy needle N1 in the ultrasound image UI. The point to which the marker Mt is set is a first insertion target point pt1, which is a first reference point pb1. In the first embodiment, the operator sets the marker Mt by using the biopsy needle N1. The reference point setting unit 53 sets the marker Mt to an intersection where an extension line 1 of the biopsy needle N1 and a plane P cross each other as shown in FIG. 8. Incidentally, reference numeral BS indicates the body surface of the subject in FIG. 8.

The setting of the marker Mt will be described specifically. The plane P is a plane which passes through the point (the noticed portion indication point pn, second reference point pb2) where the marker Mn is set, and is orthogonal to the extension line 1 of the biopsy needle N1. The reference point setting unit 53 first specifies the position and direction of the extension line 1 of the biopsy needle N1 in the coordinate system of the three-dimensional space, based on the position and direction of the biopsy needle N1 specified by the needle position specifying unit 52.

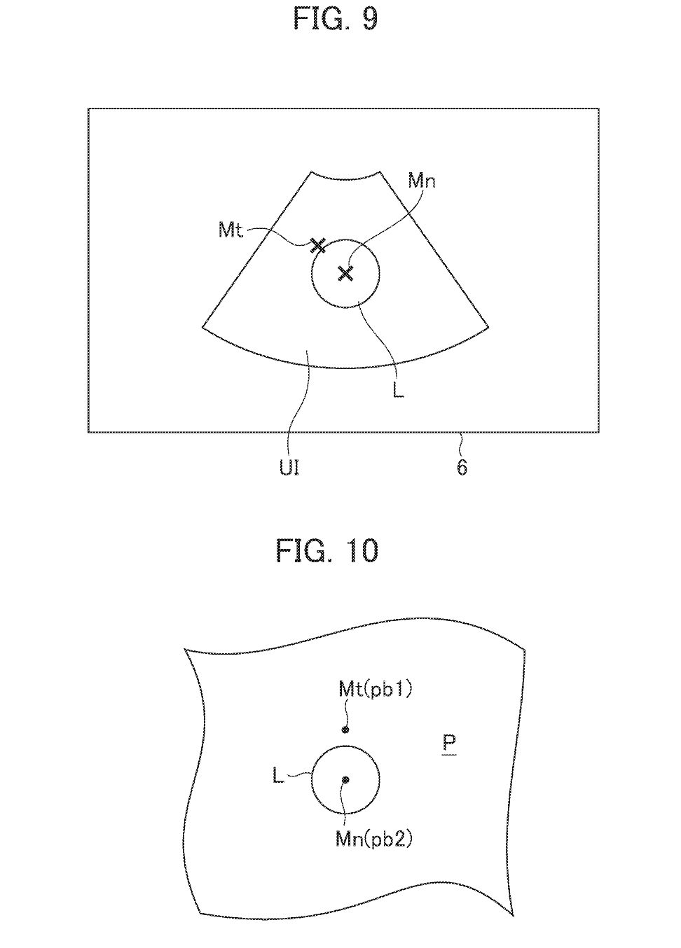

Next, the reference point setting unit 53 specifies a plane P which is orthogonal to the extension line 1 of the biopsy needle N1 and passes through the point where the marker Mn is set, in the coordinate system of the three-dimensional space, and specifies an intersection p1 between the plane P and the extension line 1 of the biopsy needle N1. Then, the reference point setting unit 53 causes the marker Mt to be displayed in the position of the intersection p1 in the ultrasound image UI as shown in FIG. 9. However, the intersection p1 is assumed to exist on the transmission/reception plane of the ultrasound.

When the marker Mt is displayed on the ultrasound image UI, a medical operator holding the biopsy needle N1 is able to easily move the marker Mt in the ultrasound image UI by changing the direction and position of the biopsy needle N1. The medical operator adjusts the direction and position of the biopsy needle N1 to place the marker Mt in a position suitable as a target point to insert the biopsy needle N1 in the ultrasound image UI.

The medical operator performs the input of determining the position of the marker Mt after the marker Mt is placed in a desired position. When this input is done, the reference point setting unit 53 determines the position of the marker Mt and does not change the position of the marker Mt even if the position and direction of the biopsy needle N1 are changed. As described above, the setting of the marker Mt to the ultrasound image UI is completed. In the first embodiment, the marker Mt is set to the outside of the lesion L and in the vicinity of the lesion L as shown in FIG. 9.

When the marker Mt is set, the reference point setting unit 53 specifies the coordinates of the marker Mt in the coordinate system of the three-dimensional space. The coordinates may be stored in the storage unit 9.

At Step S3, when the marker Mt is set to the first insertion target point pt1, the flow chart proceeds to a process of Step S4. At Step S4, the insertion target point setting unit 54 sets insertion target points other than the first insertion target point Pt1 on the basis of the marker Mt and the marker Mn set at Step S2. In the first embodiment, at Step S4, a second insertion target point pt2 is set as an insertion target point for the biopsy needle N2. A third insertion target point pt3 is set as an insertion target point for the biopsy needle N3.

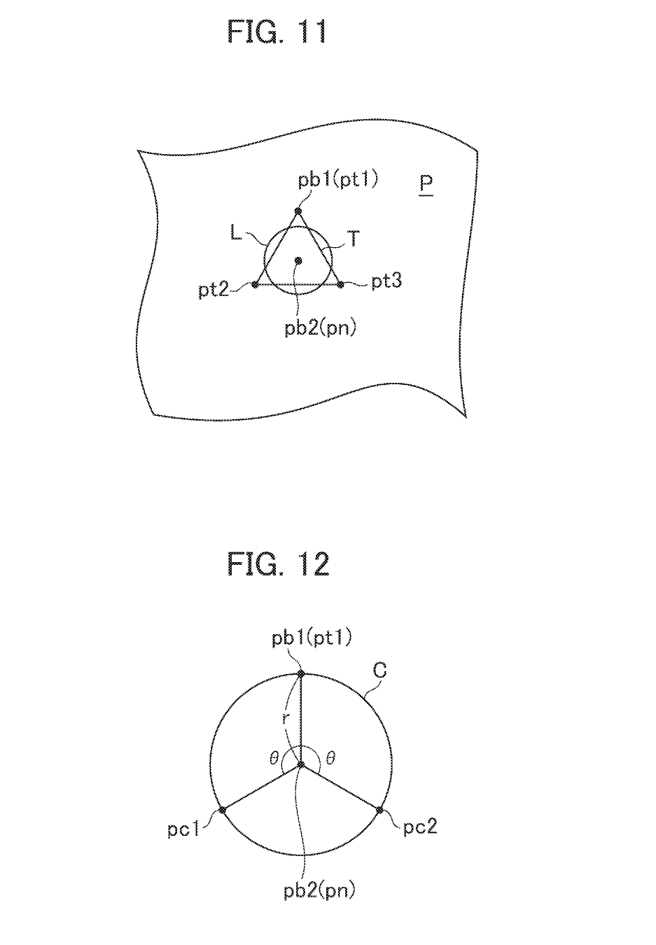

The setting of the second insertion target point pt2 and the third insertion target point pt3 will be described specifically. The insertion target point setting unit 54 sets an equilateral triangle T of which one apex is of the first reference point pb1, on the basis of the point to which the marker Mt is set, i.e., the first reference point pb1 and the point to which the marker Mn is set, i.e., the second reference point pb2. FIG. 10 is a diagram showing the first reference point pb1 (the first insertion target point pt1) and the second reference point pb2 (the noticed portion indication point pn) in the plane P. The insertion target point setting unit 54 sets the equilateral triangle T in the plane P as shown in FIG. 11. The apexes of the equilateral triangle T are the second insertion target point pt2 and the third insertion target point pt3. In the equilateral triangle T, the three apexes thereof are located around the lesion L.

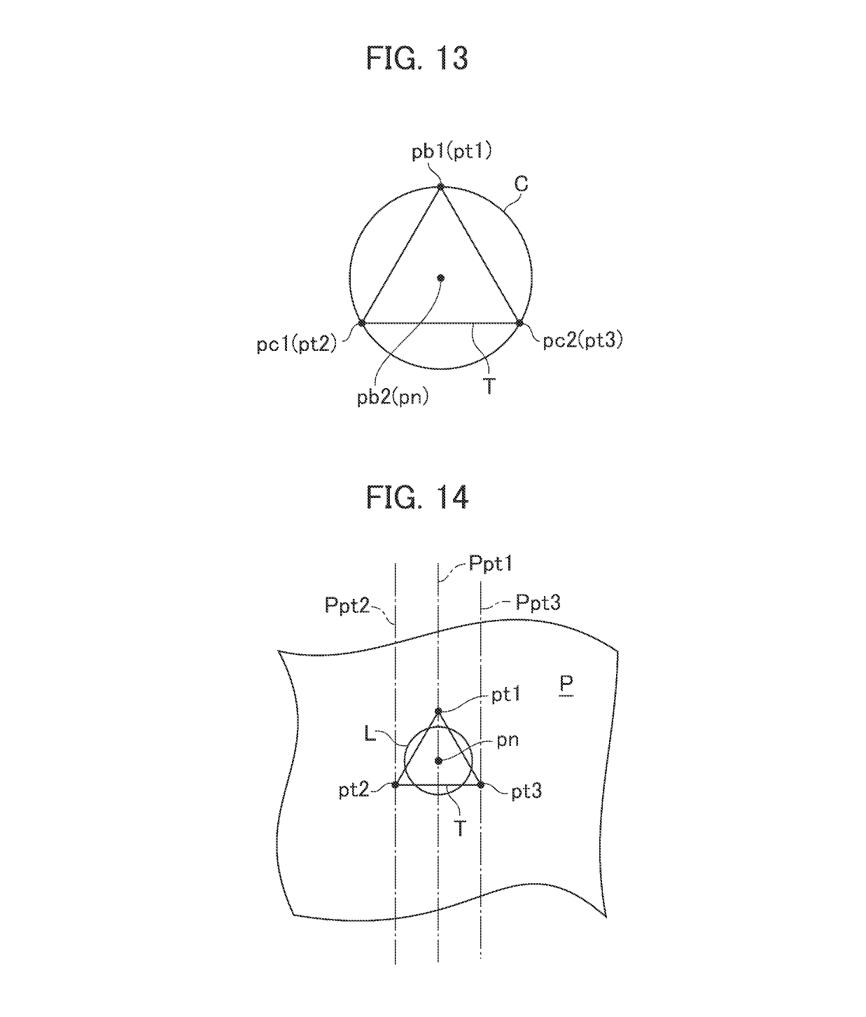

The setting of the equilateral triangle T will be described in more detail. The insertion target point setting unit 54 first sets, as shown in FIG. 12, a circle C with a distance between the first reference point pb1 and the second reference point pb2 taken as a radius r and the second reference point pb2 taken as a center within a plane P (not shown in FIG. 12). Next, the insertion target point setting unit 54 specifies points pc1 and pc2 on the circumference of the circle C of which the central angle θ is 120° with respect to a line segment s connecting the first reference point pb1 and the second reference point pb2. The points pct and pc2 on the circumference are the apexes of the equilateral triangle T and correspond to the second insertion target point pt2 and the third insertion target point pt3, as shown in FIG. 13.

Here, since the first reference point pb1 and the second reference point pb2 are respectively of the points to which the marker Mt and the marker Mn are set, the coordinates in the coordinate system of the three-dimensional space are specified. Since the second insertion target point pt2 and the third insertion target point pt3 are set on the basis of the first reference point pb1 and the second reference point pb2 as described above, the insertion target point setting unit 54 specifies the coordinates of the second insertion target point pt2 and the third insertion target point pt3 in the coordinate system of the three-dimensional space, based on the coordinates of the first reference point pb1 and the second reference point pb2. The coordinates of the second insertion target point pt2 and the third insertion target point pt3 may be stored in the storage unit 9.

Since the first reference point pb1 is located outside the lesion L, the second insertion target point pt2 and the third insertion target point pt3 being the points on the circumference of the circle C are located outside the lesion L and set in the vicinity of the lesion L. Thus, the first insertion target point pt1, the second insertion target point pt2 and the third insertion target point pt3 are set so as to surround the lesion L. By inserting the biopsy needles N1, N2 and N3 in the first insertion target point pt1, the second insertion target point pt2 and the third insertion target point pt3 set to such positions as will be described later, the biopsy needles N1, N2 and N3 can be arranged in their corresponding positions where the lesion L can be ablated thoroughly.

The operator sets the first reference point pb1 and the second reference point pb2 in such a manner that the second insertion target point pt2 and the third insertion target point pt3 are set to their corresponding positions where the lesion L can be ablated thoroughly by the biopsy needles N2 and N3.

When the second insertion target point pt2 and the third insertion target point pt3 are set at Step S4, at Step S5, the operator inserts the biopsy needles N1, N2 and N3 in the subject and performs radiofrequency-based ablation through these biopsy needles N1, N2 and N3. The biopsy needle N1 is inserted so as to pass through the first insertion target point pt1 . Further, the biopsy needle N2 is inserted so as to pass through the second insertion target point pt2. Furthermore, the biopsy needle N3 is inserted so as to pass through the third insertion target point pt3.

When the biopsy needle N1 is inserted, as shown in FIG. 14, the operator performs the transmission/reception of ultrasound in such a manner that a plane Ppt1 passing through the first insertion target point pt1 becomes a transmission/reception plane, and causes an ultrasound image UI relative to the transmission/reception plane to be displayed. When the biopsy needle N2 is inserted, the operator performs the transmission/reception of ultrasound in such a manner that a plane Ppt2 passing through the second insertion target point pt2 becomes a transmission/reception plane, and causes an ultrasound image UI relative to the transmission/reception plane to be displayed. When the biopsy needle N3 is inserted, the operator performs the transmission/reception of ultrasound in such a manner that a plane Ppt3 passing through the third insertion target point pt3 becomes a transmission/reception plane, and causes an ultrasound image UI relative to the transmission/reception plane to be displayed.

The planes Ppt1, Ppt2 and Ppt3 are assumed to be planes orthogonal to the plane P herein.

The distance indicator display control unit 55 causes the first distance indicator In1, second distance indicator In2 and third distance indicator In3 to be displayed on the ultrasound image UI as shown in FIG. 15. The first distance indicator In1 indicates a distance d1 between the first insertion target point pt1 and the transmission/reception plane of the ultrasound. The second distance indicator In2 indicates a distance d2 between the second insertion target point pt2 and the transmission/reception plane of the ultrasound. The third distance indicator In3 indicates a distance d3 between the third insertion target point pt3 and the transmission/reception plane of the ultrasound.

The distance indicator display control unit 55 calculates the distances d1, d2 and d3, based on the positions of the first insertion target point pt1, the second insertion target point pt2 and the third insertion target point pt3 in the coordinate system of the three-dimensional space and the position of the transmission/reception plane of the ultrasound in the coordinate system of the three-dimensional space. The distance indicator display control unit 55 causes the first distance indicator In1, the second distance indicator In2 and the third distance indicator In3 to be displayed based on the distances d1, d2 and d3.

The first distance indicator In1, the second distance indicator In2 and the third distance indicator In3 are respectively squares having areas corresponding to the distances d1, d2 and d3. The first distance indicator In1, the second distance indicator In2 and the third distance indicator In3 become larger in area as the distances d1, d2 and d3 increase and become smaller in area as the distances d1, d2 and d3 decrease.

When the distances d1, d2 and d3 become zero and the ultrasound transmission/reception plane coincide with the first insertion target point pt1, the second insertion target point pt2 and the third insertion target point pt3, the first distance indicator In1, the second distance indicator In2 and the third distance indicator In3 respectively become a cross shape (“+”). There is shown in FIG. 16, for example, an ultrasound image UI placed in a state in which the distance d1 becomes zero and the first insertion target point pt1 exists on the transmission/reception plane of the ultrasound. In FIG. 16, the first distance indicator In1 is in the form of a cross shape.

The positions to display the first distance indicator In1, the second distance indicator In2 and the third distance indicator In3 will be explained. When the first insertion target point pt1 exists in the transmission/reception plane of the ultrasound, the first distance indicator In1 is displayed in the position of the first insertion target point pt1 in the ultrasound image UI. On the other hand, when the first insertion target point pt1 does not exist in the transmission/reception plane of the ultrasound, the first distance indicator In1 is displayed in the position where the first insertion target point pt1 is projected onto the transmission/reception plane of the ultrasound in the ultrasound image UI.

As with the display position of the first distance indicator In1, the position to display the second distance indicator In2 is also the position of the second insertion target point pt2 or the position where the second insertion target point pt2 is projected on the transmission/reception plane of the ultrasound in the ultrasound image UI. As with the display positions of the first distance indicator In1 and the second distance indicator In2, the position to display the third distance indicator In3 is also the position of the third insertion target point pt3 or the position where the third insertion target point pt3 is projected on the transmission/reception plane of the ultrasound in the ultrasound image UI.

When the first distance indicator In1, the second distance indicator In2 and the third distance indicator In3 are respectively brought to the cross shape, a point of intersection of the cross shape corresponds to each of the first insertion target point pt1, the second insertion target point pt2 and the third insertion target point pt3.

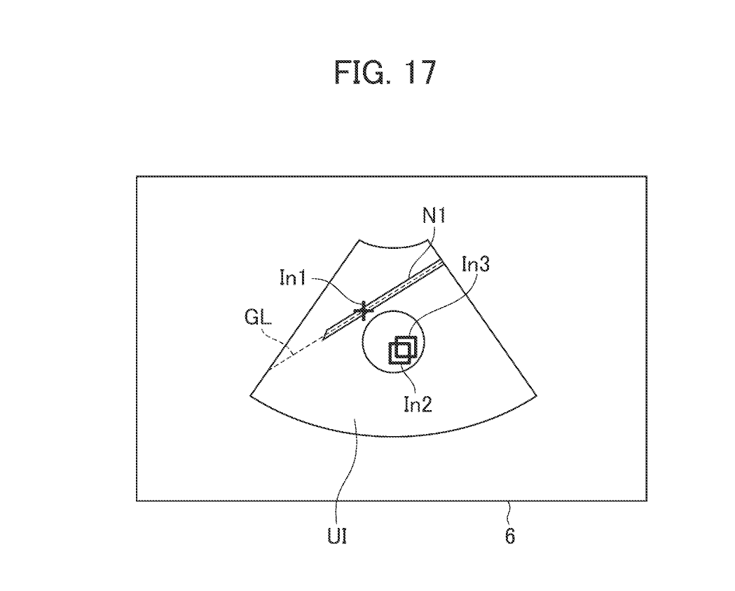

The operator moves the ultrasound probe 2 in such a manner that any of the first distance indicator In1, the second distance indicator In2 and the third distance indicator In3 is brought to the cross shape, to display an ultrasound image UI relative to any of the planes Ppt1, Ppt2 and Ppt3. Then, when the ultrasound image UI relative to any of the planes Ppt1, Ppt2 and Ppt3 is displayed, the operator inserts the biopsy needle N in the subject along the transmission/reception plane (each of the planes Ppt1, Ppt2 and Ppt3) on which the ultrasound image UI is displayed. For example, the ultrasound image UI relative to the plane Ppt1 is shown in FIG. 17. The operator inserts the biopsy needle N1 in the first insertion target point pt1 along the plane Ppt1 while viewing the ultrasound image UI. A biopsy guide line GL may be displayed on the ultrasound image UI. In this case, the operator inserts the biopsy needle N1 along the biopsy guide line GL. The operator inserts the biopsy needle N1 up to the position to allow the point thereof to pass through the first insertion target point pt1 by a predetermined distance, for example.

Although not illustrated in particular, even as for the biopsy needles N2 and N3, the operator moves the ultrasound probe 2 such that the second distance indicator In2 and the third distance indicator In3 respectively become a cross shape, to display an ultrasound image UI relative to each of the planes Ppt2 and Ppt3. Then, the operator inserts the biopsy needle N2 toward the second distance indicator In2 along the plane Ppt2, and inserts the biopsy needle N3 toward the third distance indicator In3 along the plane Ppt3.

Since the first distance indicator In1, the second distance indicator In2 and the third distance indicator In3 each cross-shaped are respectively displayed in the positions of the first insertion target point ptl, the second insertion target point pt2 and the third insertion target point pt3 in the ultrasound image UI.

When the biopsy needles N1 through N3 are inserted in the subject, radiofrequency-based ablation is performed in a state in which the three biopsy needles N1 through N3 have been inserted.

According to the first embodiment, as shown in FIG. 11 above, the first insertion target point pt1, the second insertion target point pt2 and the third insertion target point pt3 are set to the apexes of the equilateral triangle T in the plane P. Thus, if the operator inserts the biopsy needles N1, N2 and N3 toward the cross-shaped first insertion target point ptl, second insertion target point pt2 and third insertion target point pt3 in the ultrasound image UI, the biopsy needles N1, N2 and N3 can be inserted in the lesion L without deviations of their positions.

Since the first distance indicator In1, the second distance indicator In2 and the third distance indicator In3 are displayed in the ultrasound image UI, it is possible to easily display the ultrasound image UI relative to each of the planes Ppt2, Pt2 and Ppt3 including the first insertion target point ptl, the second insertion target point pt3 and the third insertion target point pt3.

### Second Embodiment

A second embodiment will next be described. The description of items overlapped with those in the first embodiment will however be omitted.

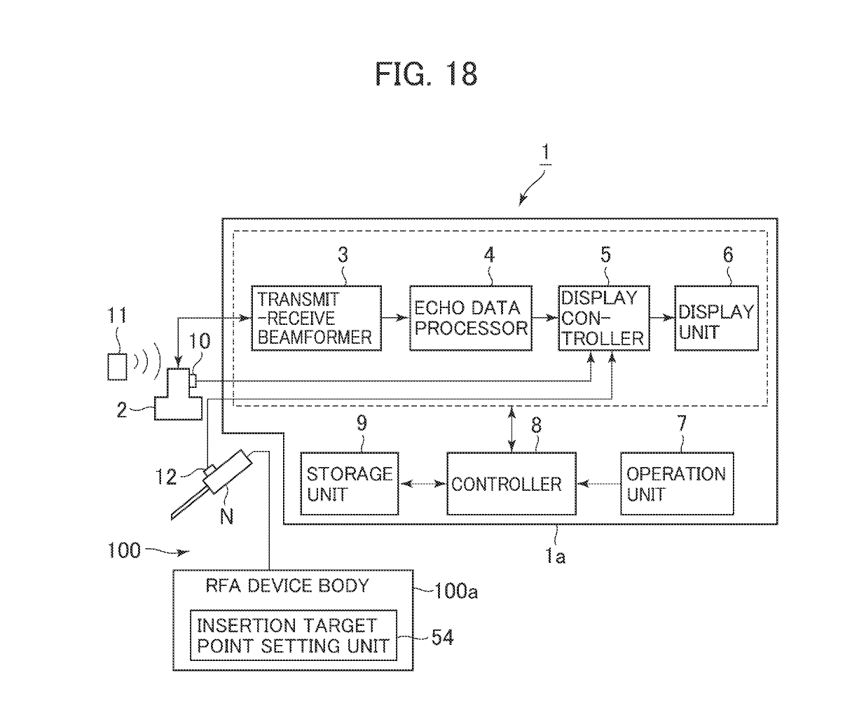

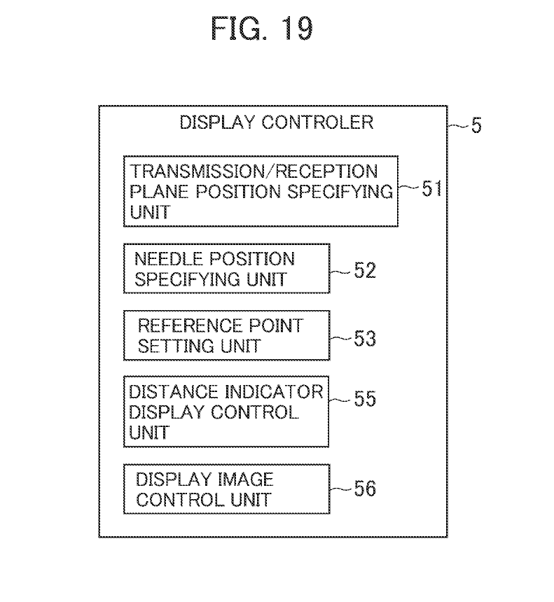

In the second embodiment, an insertion target point setting unit 54 is provided in the RFA device body 100a as shown in FIG. 18. In this case, the display controller 5 has a transmission/reception plane position specifying unit 51, a needle position specifying unit 52, a reference point setting unit 53, a distance indicator display control unit 55, and a display image control unit 56 as shown in FIG. 19. Thus, in the second embodiment, an insertion target point setting apparatus includes the ultrasound diagnostic apparatus 1 and the RFA device 100.

In the second embodiment, in order to set the second insertion target point pt2 and the third insertion target point pt3 at Step S4, the coordinates of the first reference point pb1 and the second reference point pb2 set at Steps S2 and S3 in the coordinate system of the three-dimensional space are inputted from the ultrasound diagnostic apparatus 1 to the RFA device body 100a. The insertion target point setting unit 54 of the RFA device body 100a sets the second insertion target point pt2 and the third insertion target point pt3 in the bcoordinate system of the three-dimensional space, based on the coordinates of the first reference point pb1 and the second reference point pb2 as with the first embodiment.

Information about the positions of the second insertion target point pt2 and the third insertion target point pt3 are inputted from the RFA device body 100a to the ultrasound diagnostic apparatus 1. It is thus possible to display the second distance indicator In2 and the third distance indicator In3 at Step S5.

The same advantageous effects as the first embodiment can be obtained even by the second embodiment.

Although exemplary embodiments are described herein, it is needless to say that the systems and methods described herein can be modified and implemented in various ways within the spirit and scope of the invention. For example, the insertion target points to be set are not limited to three. The insertion target point setting unit 54 draws graphics corresponding to the number of the set insertion target points and sets the insertion target points. For example, when the insertion target points to be set are four, the insertion target point setting unit 54 draws a circle which takes as a radius, the distance between the first reference point pb1 and the second reference point pb2 and has the second reference point pb2 as a center, and defines points on the circumference thereof by 90° to thereby set the apexes of a regular tetragon as insertion target points.

That is, when the number of the set insertion target points is assumed to be n, the insertion target point setting unit 54 draws a circle which takes as a radius, the distance between a first reference point pb1 and a second reference point pb2 and has the second reference point pb2 as a center, and defines points on the circumference thereof by 360°/n to thereby set the apexes of a regular n-sided polygon as insertion target points.

Further, the operator may set the first insertion target point pt1 in the ultrasound image UI using the pointing device or the like of the operation unit 7 without using the biopsy needle N.

Furthermore, the first insertion target point pt1 may be set by inserting the biopsy needle N in the subject. Specifically, the needle position specifying unit 52 specifies the position of the point of the biopsy needle N inserted in the subject, and the reference point setting unit 53 may set the first insertion target point pt to this position.

Many widely different embodiments may be configured without departing from the spirit and the scope of the present invention. It should be understood that the present invention is not limited to the specific embodiments described in the specification, except as defined in the appended claims.

1. An insertion target point setting apparatus comprising:
a reference point setting unit configured to set, in coordinates formed in a three-dimensional space with a subject with a biopsy needle inserted therein, a first reference point which is an insertion target point of the biopsy needle in the subject, and a second reference point which is a noticed portion indication point indicative of a noticed portion in the subject; and
an insertion target point setting unit configured to set other insertion target points other than the first reference point in the coordinates formed in the three-dimensional space based on the first reference point and the second reference point, using a predetermined drawing method.

2. The insertion target point setting apparatus according to claim 1, wherein the insertion target point setting unit is configured to set, based on the first reference point and the second reference point, a regular polygon of which apexes are located around the noticed portion and having as one apex the first reference point, and configured to set said other insertion target points to other apexes of the regular polygon.

3. The insertion target point setting apparatus according to claim 1, wherein at least one of first reference point, and the noticed portion indication point is set to an ultrasound image of the subject.

4. The insertion target point setting apparatus according to claim 2, wherein at least one of the first reference point, and the noticed portion indication point is set to an ultrasound image of the subject.

5. The insertion target point setting apparatus according to claim 1, wherein the first reference point is set on an extension line of the biopsy needle whose position is detected in the coordinates formed in the three-dimensional space.

6. The insertion target point setting apparatus according to claim 2, wherein the first reference point is set on an extension line of the biopsy needle whose position is detected in the coordinates formed in the three-dimensional space.

7. The insertion target point setting apparatus according to claim 3, wherein the first reference point is set on an extension line of the biopsy needle whose position is detected in the coordinates formed in the three-dimensional space.

8. The insertion target point setting apparatus according to claim 1, wherein the insertion target point setting unit is configured to set said other insertion target points on the circumference of a circle having the second reference point as a center and having as a radius, a distance between the second reference point and the first reference point.

9. The insertion target point setting apparatus according to claim 2, wherein the insertion target point setting unit is configured to set said other insertion target points on the circumference of a circle having the second reference point as a center and having as a radius, a distance between the second reference point and the first reference point.

10. The insertion target point setting apparatus according to claim 3, wherein the insertion target point setting unit is configured to set said other insertion target points on the circumference of a circle having the second reference point as a center and having as a radius, a distance between the second reference point and the first reference point.

11. An ultrasound diagnostic apparatus including an insertion target point setting apparatus according to claim 1.

12. An ultrasound diagnostic apparatus including an insertion target point setting apparatus according to claim 2.

13. An ultrasound diagnostic apparatus including an insertion target point setting apparatus according to claim 3.

14. An ultrasound diagnostic apparatus including an insertion target point setting apparatus according to claim 5.

15. An ultrasound diagnostic apparatus including an insertion target point setting apparatus according to claim 8.

16. The ultrasound diagnostic apparatus according to claim 11, including an indicator display control unit configured to cause an indicator to be displayed in a position of each of the insertion target points in the ultrasound image of the subject.

17. The ultrasound diagnostic apparatus according to claim 11, including a distance indicator display control unit configured to cause a distance indicator indicative of a distance between each of the insertion target points and a transmission/reception plane of ultrasound to and from the subject to be displayed on the ultrasound image of the subject.

18. The ultrasound diagnostic apparatus according to claim 17, wherein the distance indicator display control unit is configured to calculate the distance, based on the position of the transmission/reception plane in the three-dimensional space and the position of each of the insertion target points in the three-dimensional space.

19. The ultrasound diagnostic apparatus according to claim 18, wherein the distance indicator is displayed in a position where each of the insertion target points is projected on the transmission/reception plane, or the position of each of the insertion target points.

20. A method for setting an insertion target point comprising:
setting, in coordinates formed in a three-dimensional space with a subject with a biopsy needle inserted therein, a first reference point which is an insertion target point of the biopsy needle in the subject, and a second reference point which is a noticed portion indication point indicative of a noticed portion in the subject; and
setting other insertion target points other than the first reference point in the coordinates formed in the three-dimensional space based on the first reference point and the second reference point, using a predetermined drawing method.
